(19) United States
(12) Patent Application Publication
(10) Pub. No.: US 2008/0194930 A1
(43) Pub. Date: Aug. 14, 2008

(54) INFRARED-VISIBLE NEEDLE

(76) Inventors: Melvyn L. Harris, Folsom, CA (US); Toni A. Harris, Folsom, CA (US); Cameron Lewis, West Sacramento, CA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/673,326

(22) Filed: Feb. 9, 2007

(51) Int. Cl. A61B 5/00; H04N 5/33

(52) U.S. Cl. 600/310; 348/164; 348/E05.09

(57) ABSTRACT

A system may include an imaging apparatus and an access device. The imaging apparatus may include a light source that emits at least light having one or more wavelengths in the range of about 700 nanometers to about 2,500 nanometers, a camera that is (a) sensitive to light having a wavelength in the range of about 700 nanometers to about 2,500 nanometers, and (b) so positioned relative to the light source as to receive light having a wavelength in the range of about 700 nanometers to about 2,500 nanometers that has been (i) emitted from the light source and (ii) reflected from a target, a processor that forms an image signal based at least in part upon signals indicative of the light having a wavelength in the range of about 700 nanometers to about 2,500 nanometers that is sensed by the camera, and a display screen so coupled to the processor as to receive the image signal and to display an image. The access device may include a hollow, stiff, steel needle having an outer surface, at least a portion of which outer surface is (a) coated with a coating comprising at least one of (i) titanium nitride, (ii) gold, and (iii) a metal oxide, and/or (b) irregularized.

### SUMMARY

A system may include an imaging apparatus and a vascular access device. The imaging apparatus may include a light source that emits at least light having one or more wavelengths in the range of about 700 nanometers to about 2,500 nanometers, a camera that is (a) sensitive to light having a wavelength in the range of about 700 nanometers to about 2,500 nanometers, and (b) so positioned relative to the light source as to receive light having a wavelength in the range of about 700 nanometers to about 2,500 nanometers that has been (i) emitted from the light source and (ii) reflected from a target, a processor that forms an image signal based at least in part upon signals indicative of the light having a wavelength in the range of about 700 nanometers to about 2,500 nanometers that is sensed by the camera, and a display screen so coupled to the processor as to receive the image signal and to display an image. The vascular access device may include a hollow, stiff, steel needle having an outer surface, at least a portion of which outer surface is (a) coated with a coating comprising at least one of (i) titanium nitride, (ii) gold, and (iii) a metal oxide, and/or (b) irregularized.

### BRIEF DESCRIPTION OF THE DRAWINGS

The subject matter described below refers to the accompanying drawings, of which:

FIG. 1 depicts a basic layout of an imaging apparatus and a vascular access device.

FIG. 2 depicts an exemplary image displayed to a user by the imaging apparatus.

FIG. 3 depicts a schematic diagram of an imaging apparatus and the vascular access device with various surface coatings.

FIG. 4 schematically depicts light reflecting from a smooth surface.

FIG. 5 schematically depicts light reflecting from an example of an irregularized surface.

FIG. 6 illustrates an embodiment of a vascular access device with alternating zones of coated surfaces.

FIG. 7 illustrates an embodiment of a vascular access device having a tip coated with near infrared light reflective material.

FIG. 8 depicts a schematic diagram of an imaging apparatus and the vascular access device with an irregularized outer surface.

FIG. 9 illustrates one method of creating an irregular surface of the vascular access device.

FIG. 10 illustrates a flexible guide that can be used to create irregular surface of the vascular access device.

FIG. 11 depicts a vascular access device with an irregular surface created by a flexible guide illustrated in FIG. 10.

FIG. 12 is a perspective view of an imaging apparatus system that can rest on or be fixed to a surface, such as a table, a lamp, as a bed, or a stand such as an IV pole.

FIG. 13 is a perspective view of an imaging apparatus system that can be worn as a headband.

FIGS. 14-15 illustrate an embodiment of a wearable imaging apparatus system.

FIGS. 16-18 depict views of another embodiment of a head-mounted imaging apparatus.

FIG. 19 depicts an exemplary embodiment of an imaging apparatus mounted to an IV pole.

FIG. 20 depicts an exemplary embodiment of an imaging apparatus incorporated in a table-top system.

### DETAILED DESCRIPTION

Access to a patient's vasculature is typically obtained by advancing a needle through the patient's skin, subcutaneous tissue, and vessel wall, and into the lumen of a blood vessel. The exact location of the blood vessel may be difficult to determine because it is not in the direct sight of the user attempting to gain vascular access. The user's success in placing the distal tip of the needle in the blood vessel lumen may also be difficult to determine for similar reasons.

Consequently, proper placement of hypodermic and procedural needles can be challenging. Procedural needles are used for obtaining fluids such as spinal tap and also cells for cytology and tissue for biopsy in various locations of a human body. Medical imaging modalities have been developed to help a user guide a needle into a blood vessel by exploiting the NIR reflective and/or absorptive properties of the blood and/or surrounding tissue and a needle that has been specially prepared to reflect NIR.

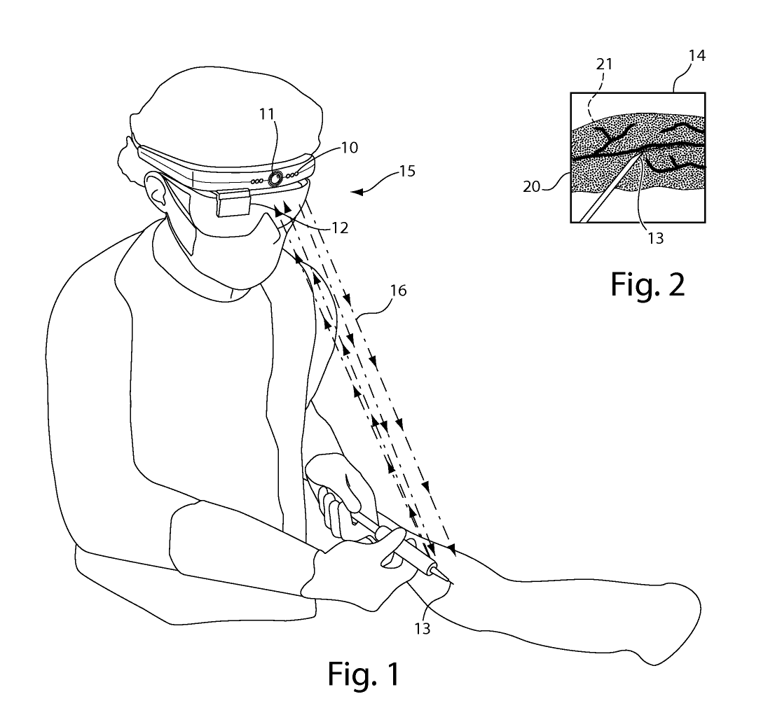

FIG. 1 depicts an imaging apparatus 15 and a vascular access device 13. The imaging apparatus 15 includes a light source 10 emitting light 16 having wavelength(s) in the range of about 700 nanometers to about 2,500 nanometers and a camera 11 which receives the light that has been emitted from the light source 10 and reflected from a target, such as the vascular access device 13 and/or tissue 20 surrounding a blood vessel 21. Also shown in FIG. 1 is a display screen 12 coupled to an image signal processor (not shown) that receives an image signal from the processor and displays an image that is visible to the human eye; an example of an image 14 that might be shown on the display screen is depicted in FIG. 2. The image displayed on the display 12 is of a real time near infrared (NIR) imaging modality revealing the location of veins beneath the skin and the contrasting position of the NIR reflective needle.

The light source 10 may include one or more light-emitting devices, such as light-emitting diode(s) or incandescent lamp(s), among others. A dedicated light source may be omitted or supplemented by ambient light, such as daylight, sunlight, or artificial light. Artificial light may be provided by incandescent lamps or (with perhaps less efficiency) by NIR-producing fluorescent lamps.

The light source may emit polarized or nonpolarized light. The imaging apparatus may include a diffuser or other device (not shown) which increases the spread of the light emitted by the light source, thereby facilitating even illumination of an anatomic site that may be positioned only inches or feet from the light source.

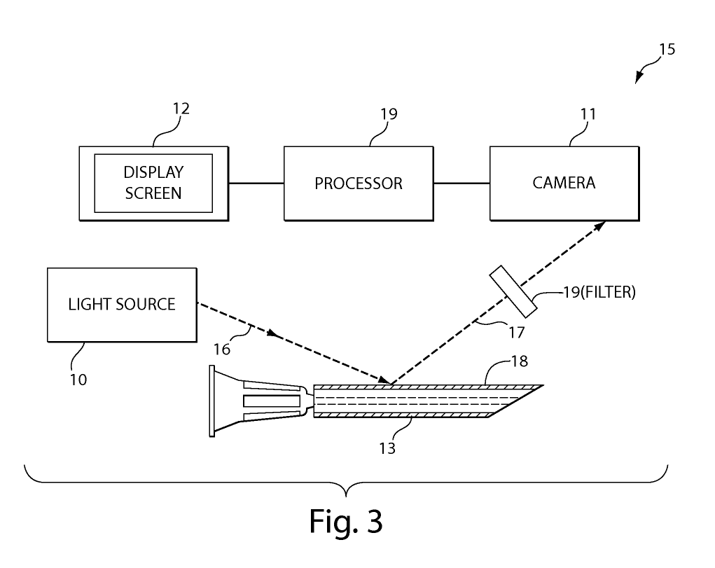

The imaging apparatus can also include one or more filters (shown in FIG. 3) to receive light reflected from the target. A wide range of filters may be employed to facilitate the passage of those portions of a light signal most useful to the visualization process. For example, a filter may be a polarizing filter. A filter may be a band-pass filter that passes light having wavelength(s) in the range of about 700 nanometers to about 2,500 nanometers to the camera 11 and removes other light. A filter may be a cut-off filter that passes light having wavelength(s) longer than about 700 nanometers and/or that attenuates light having a wavelength shorter than 700 nanometers. Exemplary cut-off filters include Wratten filters, such as Wratten filter numbers 89B, 87C, 29, 24, 25, and/or 26, among others.

A display image may be presented to a user in black-and-white, shades of gray, and/or pseudocolor. For example, the image may be rendered with bright background and dark features (black on yellow, black on white, blue on white, etc.), or bright features on a dark background. A processor may interpret light intensity signals to assign a false color or shade to a feature or to background to improve visibility. For example, in a typical application, an image of a NIR-reflective vascular access device in the vicinity of a blood vessel embedded in tissue will appear under NIR to show the device bright (high light intensity), the blood vessel dark (low light intensity), and tissue in between (medium light intensity). A processor may be programmed with intensity thresholds that instruct it, for example, to assign a first color to signals above a first threshold, a second color to signals below a second threshold, and a third color to signals between the thresholds.

A wide variety of cameras may be used, such as a charge-coupled device (CCD), complementary metal-oxide-semiconductor (CMOS), infrared-sensitive cameras, and near infrared-sensitive cameras. Two or more cameras may be used in order to generate depth information. For example, two cameras may be so positioned such that their respective images provide a stereoscopic image for a user viewing them.

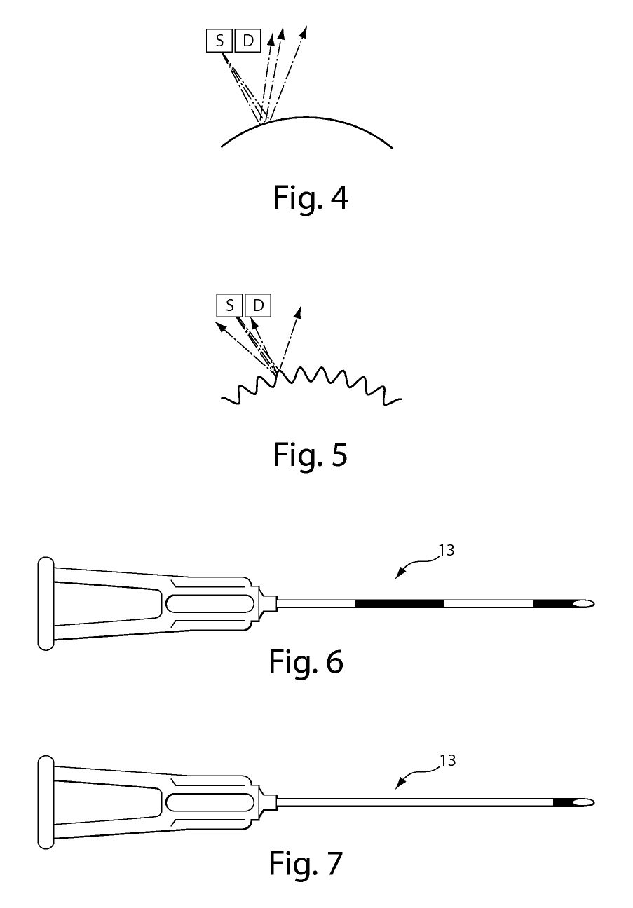

FIG. 3 illustrates a schematic diagram of the imaging apparatus 15 being used with a vascular access device 13 shown in cross-section (such as a needle, which may be hollow, stiff, and/or made of steel, such as stainless steel) having an outer surface 18 coated with a NIR reflective material. Coating the outer surface of the vascular access device 13 with an NIR reflective material enables the emitted infrared lights to reflect uniformly, thereby allowing the camera to better track the location of the vascular access device both above and beneath the skin. Current hypodermic needles are made of highly polished steel and have a smooth homogenous surface. NIR light reflected from that smooth surface tends to reflect at a precise angle or at a narrow range of angles because the incident light strikes the surface at the same or nearly the same angle and so is all reflected at the same or nearly the same angle and typically with minimal scatter (FIG. 4). Because of the minimal scatter, the polished needle reflects NIR back toward the source only at a certain angle or in a narrow range of angles. Consequently, a user might perceive the needle only if the camera happens to be positioned within that certain angle or narrow range. To compensate, a user might have to shift the camera's line of sight in the hopes of catching some reflection from the access device.

This problem may be overcome by creating irregularities in the surface of the access device. The irregularities cause the incident light to strike the surface at many different angles and, consequently, to reflect more broadly or diffusely, i.e., at many different angles, and with considerable backscatter (FIG. 5). A user is therefore much more likely to see the access device from any angle in the viewing field without repositioning the detector. So providing an access device with surface properties that vary over the surface can greatly improve NIR light reflectivity from the access device, thereby increasing visibility of the needle to the camera and permitting precise real time NIR visualization of a vein underneath the skin and the relative position of the vascular access device to the user.

The outer surface of a vascular access device 13 can be coated with at least one of titanium nitride, gold, and metal oxide. For example, metal oxide may comprise of FeOOH, CO₂TiO₄, Fe₂TiO₄, (Fe, Cr)₂O₃ and titanium oxide may include TiO₂. The surface coatings disclosed herein may be applied through a wide variety of processes including electroplating and anodizing, physical vapor deposition, chemical vapor deposition, radiation curing using an electron beam, ultraviolet and visible light, and reactive growth techniques such as annealing. In the case of gold, the coating may have a thickness in the range of 0.1 micrometers to about 100 micrometers, from 0.1 micrometers to about 10 micrometers, from 0.1 micrometers to about 5 micrometers, less than 20 micrometers, less than 10 micrometers, and/or less than 5 micrometers.

As shown in FIG. 6, a needle may have alternating regions of coated sections to allow the observer to gauge the length of the needle 13 under the imaging apparatus. For example, coated bands can be spaced apart in one centimeter intervals so a user can gauge the location and/or depth of the needle. These gaps can be created at different increments using various NIR reflective materials or surface irregularization.

The irregularities in the surface may be regular (i.e., periodic or patterned, such as a smoothly undulating surface) or irregular (aperiodic, random, or pseudorandom, such as a spray coating or roughening).

In some embodiments, the coating thickness may vary at different positions along the length of the outer surface of the needle 13. Thickness variations in the coating may help create facets of the coating facing many different directions; they may improve the scattering of NIR light from the coating, thereby improving the needle's visibility.

In some embodiments, the coating may be nonhomogeneous; i.e., includes different amounts of substances in different regions of the coating. In this way, NIR reflectivity intensity and/or direction may vary from region to region, thereby increasing visibility of the vascular access device. A nonhomogeneous coating may be provided by, for example, incompletely mixing component parts (such as two batters may be incompletely mixed to make a marble cake), or by applying a coating in multiple layers, with one layer applied to certain regions and a second layer applied to other, but possibly overlapping, regions.

As shown in FIG. 7, a coating (and/or surface irregularization) may be applied only to the tip of the hollow shaft to localize NIR reflectivity. In this configuration, the needle can serve as a trocar or an introducer for a catheter that houses the needle. Previously discussed needle configurations can also serve the same purpose as long as the tip of the needle, at least, is coated with a NIR reflective material and/or irregularized.

In some embodiments, a needle and/or guidewire having enhanced NIR reflectivity (as by coating and/or irregularization) may be used as a guide for advancement of another device, such as a catheter. A portion of the needle ad/or guidewire surface, or all of it, may be treated. The, e.g., catheter may be NIR transparent, or at least have regions of NIR transparency, so that the needle and/or guidewire may be seen by a user when the catheter slides over it. The, e.g., catheter may itself have one or more NIR-reflective features so that its position relative to the needle and/or guidewire can be appreciated.

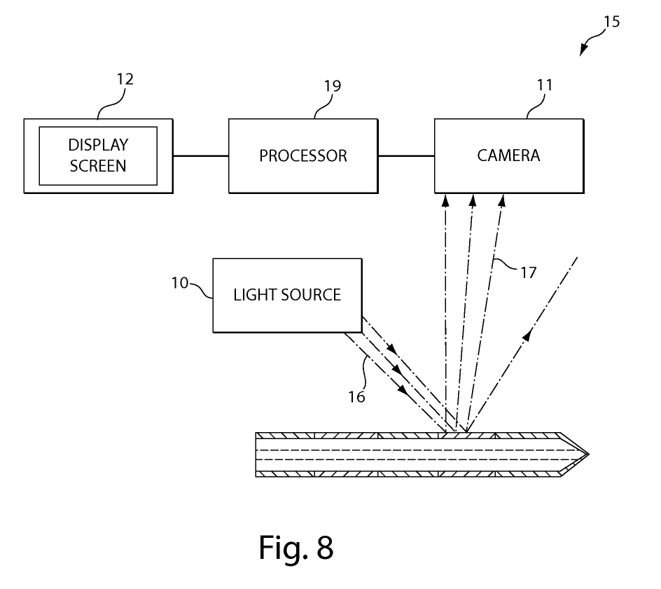

In some embodiments, at least a portion of the outer surface of the vascular access device 13 is irregularized as to reflect light in the range of about 700 nanometers to about 2,500 nanometers at all or substantially all possible angles as shown in FIG. 8. The term “irregularized” as used herein refers to a modification or inherent manufacturing feature of a needle that makes its surface less smooth than that of a standard stainless-steel needle. One advantage of an irregularized surface over a smooth homogenous surface is maximized scatter of reflected NIR, as discussed previously. Conventional hypodermic needles are made of a highly polished steel which yields inconsistent reflection under NIR imaging devices. The entire surface of the vascular access device may be irregularized. For example, in the case of a steel needle, the entire needle surface may be irregularized, such as by acid etching.

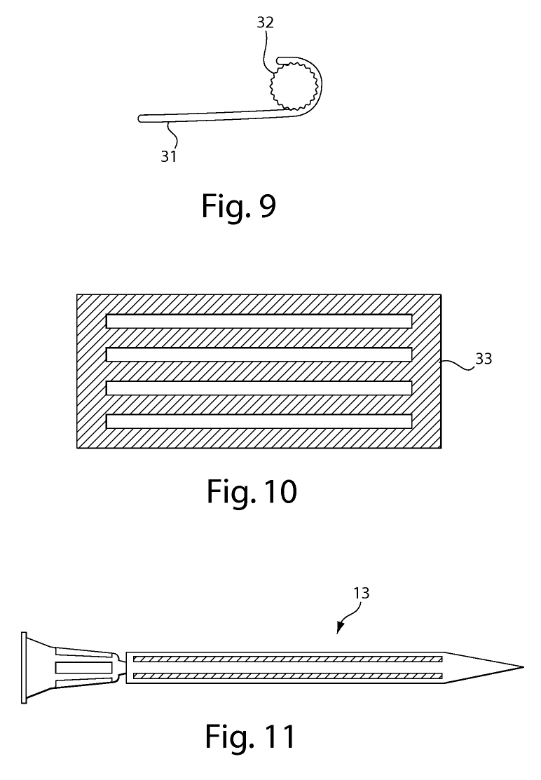

FIG. 9 illustrates a vascular access device 13 having an irregularized outer surface by rolling a sheet metal 31 onto an irregularly shaped guide 32 to create surface irregularity. Surface irregularity can also be formed by subjecting an initially smooth steel vascular access device 13 to abrasion, machining, blasting, chemical etching such as acid etching (as with, for example, nitric acid, hydrofluoric acid, hydrochloric acid, and/or sulfuric acid), or heating. For example, parts of the outer surface can be protected by a sleeve or a guide from abrasion to create alternating patterns of surface morphology. In some embodiments, the irregularized surface can be coated with NIR reflective materials in constant or varying thicknesses to maximize NIR reflectivity.

A flexible guide template 33 as shown in FIG. 10 is wrapped around the vascular access device 13 during blasting to create alternating bands of surface finish illustrated in FIG. 11. The flexible guide template 33 may also be used to coat NIR reflective material over a homogenous outer surface of the vascular access device as illustrated in FIG. 3. The shape and size of the template pattern can be varied in a number of different ways to suit varying purposes of the vascular access device 13.

In some embodiments, a needle's outer surface may both be irregularized and have a coating. The various coating and irregularization features disclosed may be combined to improve further the needle's NIR reflectivity and/or to give the needle a distinctive reflection pattern to help the user visualize it.

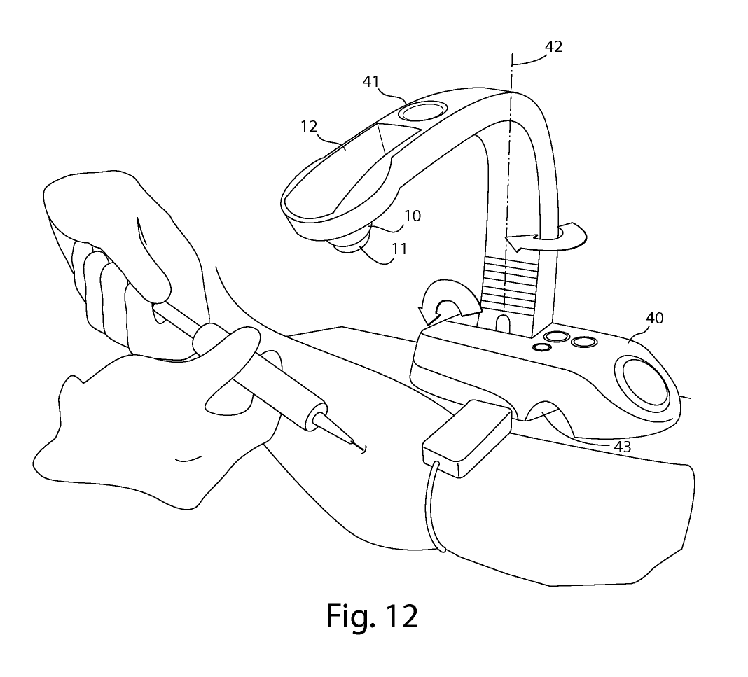

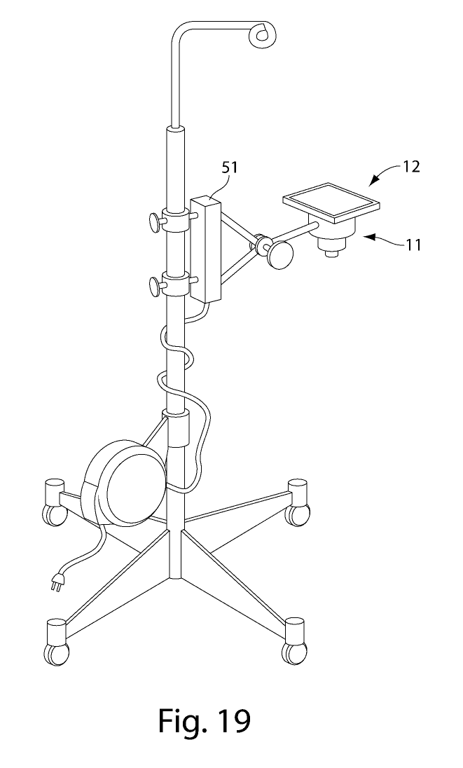

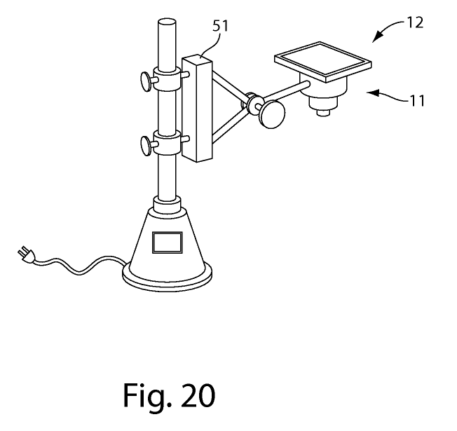

FIG. 12 depicts an embodiment in which, camera 11, light source 10, and the display screen 12 are attached to an arm 41 of base 40. The arm 41 may be curved (as shown) and has a display screen 12 on the upper surface to show a real time NIR photography revealing the location of the veins beneath the skin. The user can maintain the position of the arm relative to the base according to the user's preference since the arm 41 may bend and/or rotate about the axis 42. The length and/or the position of the arm in the imaging apparatus can be adjusted to allow optimal emission and reflection of the NIR lights. As illustrated in FIG. 12, the base 40 can be fixed to a bed rail, IV pole (FIG. 19), or other surface by mating feature 43. The system can be configured as a table-top system (FIG. 20).

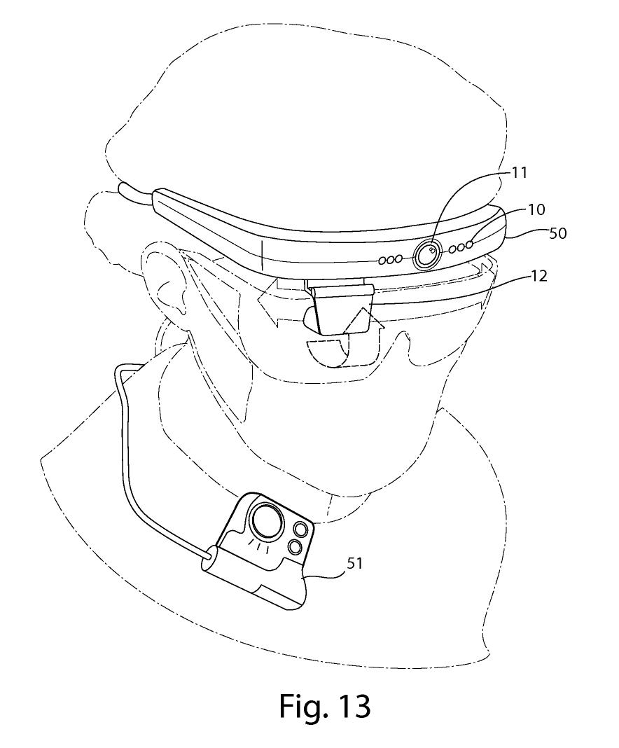

FIG. 13 illustrates an image apparatus system so sized and shaped as to be positionable on a user's head. The display screen 12 is attached to a headband 50 to allow the display screen to be substantially or completely out of the user's field of vision when desired. For example, the screen may be attached to the headband by a hinge, thereby allowing a user to flip the screen into his or her line of sight during a vascular access procedure and to flip it out view before or after such a procedure. Instead of a headband, the display screen may be mounted to a visor, glasses, or other device that may be so positioned on the head as to make the display screen positionable in the wearer's line of sight. When the screen is in the viewer's field of vision, the image it displays can be registered with the user's normal line of sight, so that the image the user perceives from the screen combines precisely with the visible-light image the user perceives through the uncovered eye. Such a combination can provide additional guidance for a user to ensure proper needle placement. The display screen 12 can also be adjusted laterally across the headband 50 for custom positioning of the display screen. Adjustable display screen allows a user to control the location of the screen so that an unobstructed on-screen view can be seen.

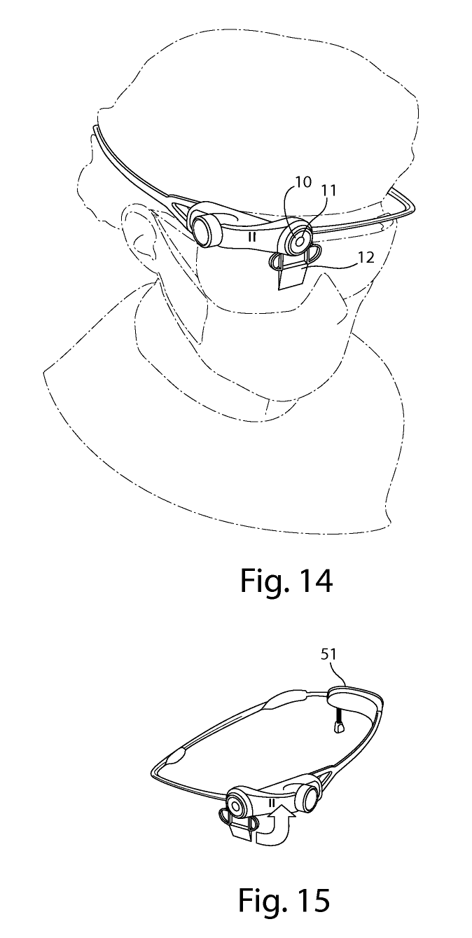

The camera 11 and/or the light source 10 may also be attached to the headband 50. In some embodiments, the camera and/or light source may be attached by a hinge to optimize alignment of the camera and the line of sight of the user. In some embodiments, the camera and the light source may be positioned such that the light source 11 encloses the camera 10 as shown in FIG. 14. The display screen 12 can also be programmed to turn on when it is flipped down and turn off when it is flipped up. A power supply 51 component, shown in FIG. 15, may either be worn on the user as illustrated or may be an integral part of the headband that a user wears. (FIGS. 13-15 depict the user as wearing a mask, but a mask is optional.)

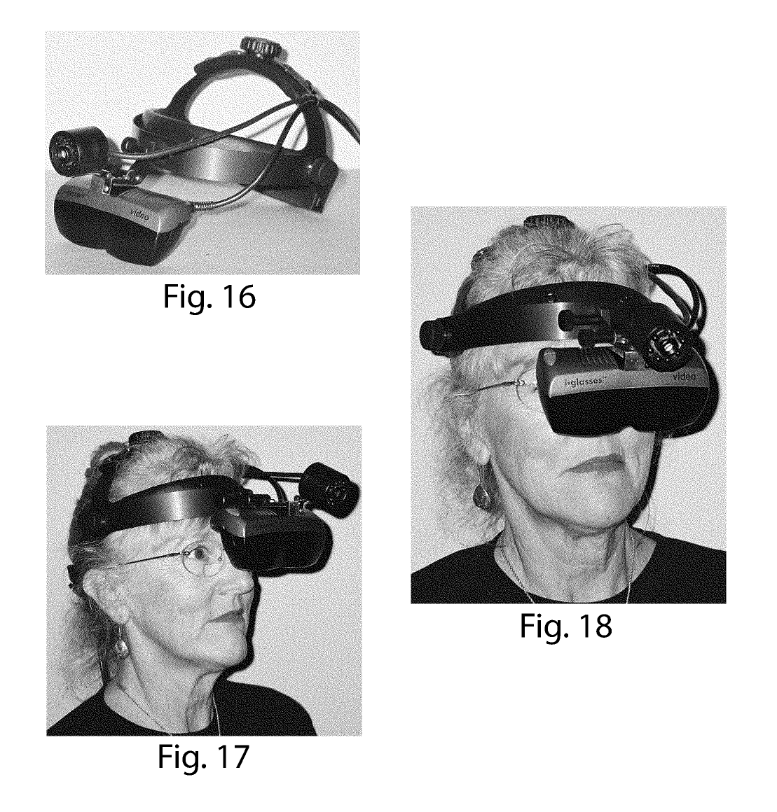

FIGS. 16-18 depict views of another embodiment of a head-mounted imaging apparatus.

Additional examples of imaging apparatus arrangements and orientations are disclosed in U.S. Pat. Nos. 6,032,070, 4,817,622, 5,608,210, and 5,519,208, and in U.S. Pat. App. Pub. Nos. 20060173351, 20050281445, 20040019280, and 20030187360, which are hereby incorporated herein by this reference. In particular, systems described in U.S. Pat. No. 6,556,858, U.S. Pat. App. Pub. Nos. 20040111030, 20060122515, and U.S. patent application Ser. No. 11/610,140, each of which is hereby incorporated herein by this reference, may be combined with a NIR-visibility-enhanced vascular access device as disclosed herein.

Other irregularization and/or coating techniques that may be employed are described, e.g., in U.S. Pat. Nos. 4,962,041, 6,749,554, 6,610,016, 6,306,094, 5,383,466, 6,178,340, 6,860,856, 4,582,061, 5,290,266, 6,970,734, 4,959,068, 4,905,695, 5,782,764, 6,176,871, 3,038,475, 3,376,075, and 5,358,491, and U.S. Pat. App. Pub. Nos. 20020115922, 20040019280, 20040260269, 20050222617, 20030187360, 20060204456, 20040254419, 20060201601, 20040267195, and 20050096698, each of which is hereby incorporated herein by this reference.

The disclosed systems may also incorporate three-dimensional (3D) imaging technology to improve further the user's target acquisition ability. Various 3D modalities are particularly well suited for use with NIR, including NIR tomography, NIR-based confocal microscopy, multispectral stereoscopy, and volumetric 3D. These and other imaging techniques are described in, e.g., U.S. Pat. Nos. 5,841,288, 6,183,088, 6,321,759, 6,448,788, 6,487,020, 6,489,961, 6,512,498, 6,554,430, 6,570,681, 6,766,184, 6,873,335, 6,885,372, 6,888,545, 6,940,653, 7,012,601, 7,023,466, 7,144,370, and 7,164,105, and in U.S. Pat. App. Pub. Nos. 20010045920, 20020065468, 20030146908, 20040064053, 20040077943, 20040135974, 20040212589, 20050152156, 20050203387, 20050213182, 20050219241, 20050230641, 20050270645, 20050285027, 20060007230, 20060012367, 20060026533, 20060028479, 20060056680, 20060092173, 20060109268, 20060241410, and 20060244918, each of which is hereby incorporated herein by reference.

Although this disclosure describes vascular access devices with enhanced NIR visibility, a wide variety of other medical devices may be similarly modified to improve their visibility during use, especially invasive devices, such as catheters, biopsy needles, ablation tips, endoscopic instruments, laparoscopic instruments, arthroscopic instruments, etc. Various structures may be targeted, particularly targets that absorb NIR, such as blood vessels, veins, arteries, central veins, central arteries, vascularized tumors, etc. The systems and methods disclosed herein may be used in a wide variety of procedures, such as obtaining vascular access, obtaining biopsies, administering therapeutic substances by injection targeted to a site, obtaining access to non-vascular spaces such as peritoneal, pleural, mediastinal, spinal, and/or gastrointenstinal spaces. The disclosed systems and methods may be used in animals, including humans and non-human animals.

We claim:

1. A system comprising:
an imaging apparatus that comprises:
a light source that emits at least light having one or more wavelengths in the range of about 700 nanometers to about 2,500 nanometers;
a camera that is (a) sensitive to light having a wavelength in the range of about 700 nanometers to about 2,500 nanometers, and (b) so positioned relative to the light source as to receive light having a wavelength in the range of about 700 nanometers to about 2,500 nanometers that has been (i) emitted from the light source, and (ii) reflected from a target;
a filter that attenuates light having a wavelength shorter than 700 nanometers and is so positioned as to (i) filter light emitted from the light source, and/or (ii) filter light reflected from the target before it is received by the camera;
a processor that forms an image signal based at least in part upon signals indicative of the light having a wavelength in the range of about 700 nanometers to about 2,500 nanometers that is sensed by the camera; and
a display screen so coupled to the processor as to receive the image signal and to display an image; and
an access device that comprises a hollow, stiff, steel needle having an outer surface, at least a portion of which outer surface is (a) coated with a coating comprising at least one of (i) titanium nitride, (ii) gold, and (iii) a metal oxide, and/or (b) irregularized.

2. The system of claim 1, wherein the light source comprises a light-emitting diode (LED).

3. The system of claim 1, wherein the light source comprises an incandescent lamp.

4. The system of claim 1, wherein the camera comprises a charge-coupled device (CCD) camera.

5. The system of claim 1, wherein the camera comprises a complementary metal-oxide-semiconductor (CMOS) camera.

6. The system of claim 1, wherein the system further comprises a base and an arm extending from the base, wherein at least one of the light source, the camera, and the display screen are disposed in or on the arm.

7. The system of claim 6, wherein at least two of the light source, the camera, and the display screen are disposed in the arm.

8. The system of claim 6, wherein the light source, the camera, and the display screen are disposed in the arm.

9. The system of claim 1, wherein the system is so sized and shaped as to be positionable on a user's head, and wherein the display screen is, in at least one orientation, positioned to be in the user's field of vision.

10. The system of claim 9, further comprising a headband to which the display screen is attached.

11. The system of claim 10, wherein the display screen is pivotally attached to the headband and is pivotable between a first orientation in which it is in the user's field of vision and a second orientation in which it is substantially or completely out of the user's field of vision.

12. The system of claim 10, wherein the camera is attached to the headband.

13. The system of claim 12, wherein the light source is attached to the headband.

14. The system of claim 1, wherein at least the portion of the needle outer surface is coated.

15. The system of claim 14, wherein the coating comprises gold.

16. The system of claim 15, wherein the gold coating has a thickness of less than 5 micrometers.

17. The system of claim 14, wherein the coating comprises a metal oxide.

18. The system of claim 17, wherein the metal oxide comprises a titanium oxide.

19. The system of claim 18, wherein the titanium oxide comprises TiO₂.

20. The system of claim 17, wherein the metal oxide comprises FeOOH.

21. The system of claim 17, wherein the metal oxide comprises Co₂TiO₄.

22. The system of claim 17, wherein the metal oxide comprises Fe₂TiO₄.

23. The system of claim 17, wherein the metal oxide comprises (Fe,Cr)₂O₃.

24. The system of claim 14, wherein the coating comprises titanium nitride.

25. The system of claim 14, wherein the portion of the needle outer surface is irregularized.

26. The system of claim 25, wherein the coating thickness varies at different positions in the needle outer surface portion.

27. The system of claim 1, wherein the portion of the needle outer surface is irregularized.

28. The system of claim 27, wherein the coating thickness varies at different positions in the needle outer surface portion.

29. The system of claim 27, wherein the irregularized portion of the needle outer surface includes regions so positioned as to reflect incident light in the range of about 700 nanometers to about 2,500 nanometers at all or substantially all possible angles.

30. The system of claim 27, wherein the irregularized portion is formed at least in part by subjecting an initially smooth steel needle to abrasion, machining, blasting, chemical etching, or heating.

31. The system of claim 27, wherein the irregularized portion is formed at least in part from sheet metal rolled against an irregular guide.

32. A method comprising:
illuminating a target to which access is to be obtained with light emitted from the light source of the imaging apparatus of the system of claim 1;
advancing the access device of claim 1 toward the target; and
while advancing, visualizing the target and the access device on the display screen.

33. The method of claim 32, wherein the target comprises a blood vessel of a subject.

34. The method of claim 32, wherein the target is located in a non-human animal subject.
